(19) United States
(12) Patent Application Publication
(10) Pub. No.: US 2025/0339055 A1
(43) Pub. Date: Nov. 6, 2025

(54) FALL PREVENTION AND TRAINING

(71) Applicant: Cochlear Limited, Macquarie University, NSW (AU)

(72) Inventors: Marita Elizabeth Neilson, Bayview, NSW (AU); Benedikt Funke, Berkeley Vale, NSW (AU); Luke Crowe, St Leonards, NSW (AU)

(21) Appl. No.: 18/867,180

(22) PCT Filed: May 23, 2023
(86) PCT No.: PCT/IB2023/055306

§ 371 Date: Nov. 19, 2024

Related U.S. Application Data
(60) Provisional application No. 63/345,961, filed on May 26, 2022.

(51) Int. Cl. A61B 5/11; A61B 5/00

(52) U.S. Cl. CPC A61B 5/112; A61B 5/4836; A61B 5/686; A61B 2505/09

(57) ABSTRACT

Presented herein are techniques for providing tailored sensory cues to assist with immediate balance correction, and/or tailored training exercises to improve natural balance. The tailored interventions via sensory cues assist with immediate balance correction and are directed to providing users with sensory cues that instruct users to correct gait or balance issues to prevent an imminent fall. The tailored interventions via training exercises are also tailored to the specific users to provide specific exercises to address specific balance or gait deficiencies of particular users.

### BACKGROUND

### Field of the Invention

The present invention relates generally to techniques for providing interventions for individuals with balance and/or gait deficiencies.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In some aspects, the techniques described herein relate to a method including: determining, from historical sensor data acquired from a user-worn sensor device, a baseline balance profile for a user; tracking, via the user-worn sensor device, balance characteristics of the user; determining, from the baseline balance profile for the user and the tracked balance characteristics of the user, a motion of the user that deviates from the baseline balance profile; and providing to the user a sensory cue to correct for the motion that deviates from the baseline balance profile.

In other aspects, the techniques described herein relate to a method including: tracking, via a user-worn sensor device, balance characteristics of a user; determining, from the balance characteristics of the user, a baseline balance profile for a user; determining, based on the baseline balance profile, an exercise for the user to improve the balance characteristics of the user; and presenting, via an electronic device associated with the user, data indicative of the exercise for the user.

In still other aspects, the techniques described herein relate to a method including: obtaining balance characteristic data for a user; determining, from the balance characteristic data, average range and maximum variation values for the balance characteristic data for a plurality of time periods; and determining, from the average range and the maximum variation values for the balance characteristic data for the plurality of time periods, a user-specific baseline balance profile for the user.

In still other aspects, one or more non-transitory computer readable storage media are provided. The one or more non-transitory computer readable storage media comprise instructions that, when executed by one or more processors, cause the one or more processors to: determine, from historical sensor data acquired from a user-worn sensor device, a baseline balance profile for a user; track, via the user-worn sensor device, balance characteristics of the user; determine, from the baseline balance profile for the user and the tracked balance characteristics of the user, a motion of the user that deviates from the baseline balance profile; and provide to the user a sensory cue to correct for the motion that deviates from the baseline balance profile.

In still other aspects, a system is provided. The system comprises: a user worn sensor device configured to acquire data associated with balance or gait characteristics of a user; a user interface device; and a processing device comprising one or more processors configured to: receive, from the user worn sensor device, first data associated with the balance or gait characteristics of the user; determine, from the first data, a baseline balance profile for a user; receive, via the user worn sensor device, second data associated with the balance or gait characteristics of the user; determine, from the baseline balance profile for the user and the second data, a motion of the user that deviates from the baseline balance profile; and provide to the user, via the user interface device, a sensory cue to correct for the motion that deviates from the baseline balance profile.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a first flowchart illustrating a holistic process flow for implementing the fall prevention techniques disclosed herein;

FIG. 3 is a second flowchart illustrating a process flow for implementing sensory cue intervention techniques disclosed herein;

FIG. 4 is third flowchart illustrating a process flow for implementing tailored training exercise intervention techniques disclosed herein;

FIG. 5 illustrates a series of steps via which a baseline balance profile for a user is generated according to the techniques disclosed herein;

FIG. 6 is a fourth flowchart illustrating a process flow for generating a baseline balance profile for a user according to the techniques disclosed herein;

FIG. 7 is schematic diagram illustrating an implantable stimulation system with which aspects of the techniques presented herein can be implemented;

FIG. 8 is a schematic diagram illustrating a vestibular stimulator system with which aspects of the techniques presented herein can be implemented; and

FIG. 9 is a schematic diagram illustrating a retinal prosthesis system with which aspects of the techniques presented herein can be implemented.

### DETAILED DESCRIPTION

Presented herein are techniques for addressing known risks, such as falls, in a portion of the of the cochlear implant recipient population (e.g., older adults). However, the disclosed techniques may be relevant to any user who suffers from balance or gait problems, not just cochlear implant recipients or older adults. The disclosed techniques may also be relevant to individuals with undiagnosed balanced issues or populations at risk of developing balance issues in the future, such as the elderly. For example, research has shown that as many as one-third of older adults may fall at least once over the course of a year. Accordingly, falls and the fear thereof may contribute to restricted activity in older populations, and such populations may avoid activity to reduce the perceived risk of falls. This decreased activity may actually increase the risk of falls due to, for example, decreased fitness. Fall-related injuries (e.g., hip fractures and head injuries) may also contribute to increasing care costs for older adults. Therefore, accurately identifying individuals requiring intervention to reduce fall risk may provide substantial benefits in older populations. As described below, the disclosed techniques may utilize user-worn sensors to detect and address balance issues, even prior to a clinical diagnosis.

The techniques presented herein provide several key interventions, including: (1) tailored sensory cue interventions to assist with immediate balance correction, and (2) tailored training exercise interventions to improve natural balance in users with balance or gait problems. Both of these interventions are provided in response to user gait or balance data gathered from user-worn sensors, such as accelerometers, gyroscopes, inclinometers, compasses, magnetometers, barometers, and the like. The data acquired from these sensors allows for the creation of a user-specific baseline balance profile. For example, a user may undergo a learning period of a few days, a week, or a few weeks, during which data indicative of the user's gait and balance are acquired from the sensors. This sensor data may then be used to generate the baseline balance profile for the user. The baseline balance profile may be used to assess the user's current gait or balance, as well as to assess changes to the user's gait and balance moving forward.

The baseline balance profile may also be used to present the user with exercises tailored to improve his or her natural balance. These tailored training exercises may include specific exercises to address specific balance or gait deficiencies for the user identified from the user baseline balance profile. Continued tracking and analysis of the user gait or balance data allows for the determination of improvement or degradation of the user's balance and/or gait, which allows the disclosed techniques to provide updated exercises to the user. The continued tracking of the user's gait and balance characteristics also facilitates providing the user with feedback indicating the effect of the exercises on the user's gait or balance, thereby motivating the user to continue making use of the exercises.

After creation of the user-specific baseline balance profile, user gait or balance data may continue to be acquired by the user-worn sensors in order to provide the user with cues intended to provide immediate or real-time instruction for the user to correct the detected issues in their balance or gait. Specifically, sensor data that deviates from the user-specific baseline balance profile may indicate that the user is likely to fall. The user may be presented with a sensory cue that prompts the user to correct his or her balance and avoid the fall. The tailored sensory cue interventions assist with immediate balance correction and are directed to providing users with sensory cues that instruct the user how to avoid an otherwise imminent fall. For example, if a user is leaning too far to the left, an auditory or haptic sensory cue may be applied to the left side of the user's body, indicating to the user the direction in which they should correct their balance.

The initially determined baseline balance profile may be maintained for the user until, for example, the baseline balance profile is no longer providing accurate balance or gait cues, either because the user's balance or gait has improved and the cues are no longer indicative of balance problems, or the user's balance or gait has degraded and the cues are not being provided in a way that prevents falls or other balance or gait issues. At such time, a new learning period may be undertaken to generate a new baseline balance profile for the user.

Unlike related art techniques, the techniques described herein tailor the sensory cues to the needs of specific users. For example, related art sensory substitution systems may provide audio cues in a manner that adversely impacts the user's hearing and cognition. Such cues may be especially troubling for a person with mild balance problems who is more likely to be socially active. In social situations, the user may find that the ongoing delivery of balance cues interferes with their attention and ability to hear conversations. Moreover, a user with mild balance problems may become habituated to the audio cues and start ignoring the cues altogether or relegate the cues to sub-conscious perception. Additionally, pre-clinical users may be less willing to tolerate such intrusive balance cues because they have not been medically diagnosed with a condition. A user with mild balance problems may be unaware of the problems, and thus have low motivation and be less tolerant of intrusive interventions. By tailoring balance cues to the specific user, inventions may be achieved that reduce fall risks in a manner that is tolerated by a population with mild or undiagnosed balance problems. Similarly, users with normal or near-normal balance when they initially receive an implant may receive balance cues if and/or when their balance degrades over time.

Both the tailored sensory cue and tailored training exercise intervention techniques disclosed herein may be guided by sensors and artificial intelligence (AI) that are working together to assess momentary and average balance and gait characteristics of the user, and thus generate fall risk assessments. The sensors utilized in the disclosed techniques may include 3-axis accelerometer sensors, which may be housed within a hearing prosthesis, a medical implant, or a dedicated sensor implant. The sensors may also be incorporated into personal electronic devices, including smartphones, smart watches, and other wearable sensor devices. The sensors utilized in the techniques may be configured to detect acceleration of less than 1 g to identify changes of its position relative to Earth's gravitational field and small movements of the user's head. Other types of sensors may also be used as supplements or alternatives to accelerometer sensors. For example, a gyroscope may be used to measure orientation and angular velocity (e.g., pitch, roll, and yaw) of a user's head. A magnetometer may be used to detect the Earth's magnetic field, allowing measurement of absolute orientation rather that relative movement. A barometric pressure sensor may be used to detect sudden changes in altitude, pointing to a fall or a change in the user's position.

A computing device may be used to gather data from the above-described sensors. For example, an application or “app” installed on a user's smartphone may receive data from the sensors and transmit the data to a server device that assesses the gait or balance data to determine balance changes and fall risks for the user. The results of the assessment may be communicated back to the smartphone application. The assessment of the gait or balance data may also be performed by the same computing device that receives the data from the sensors. Based upon the received assessments, the application may then be configured to implement the tailored sensory cue intervention techniques and the tailored training exercise intervention techniques disclosed herein.

The application may also present users with Ecological Momentary Assessments (EMAs) that can be used in the assessment of the user's gait and/or balance. EMAs refer to event-dependent questions and responses. The disclosed techniques provide for EMA questions to be presented to the user and EMA responses to be received from the user via the smartphone application. These responses may be used to, for example, determine appropriate interventions for the user, such as exercises for the user to perform to improve their gait or balance. According to one specific example, if an instability, such as a sway, is detected in the user's gait or balance (e.g., via the sensors as described above), the application may administer a question prompting the user to indicate the type of activity that had just been performed. The question may include a list of activities via a dropdown menu presented to the user via the application. The user's response to the question may be inputted into the algorithm that determines balance exercises. Accordingly, user indications of actual activity can be included in the assessments. These user-provided responses allow the algorithm to interpret the sensor signals based on actual user feedback, as well as through AI and machine learning techniques.

Merely for ease of description, the techniques presented herein are primarily described with reference to a specific implantable medical device system, namely a cochlear implant system. However, it is to be appreciated that the techniques presented herein may also be partially or fully implemented by other types of implantable medical devices. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

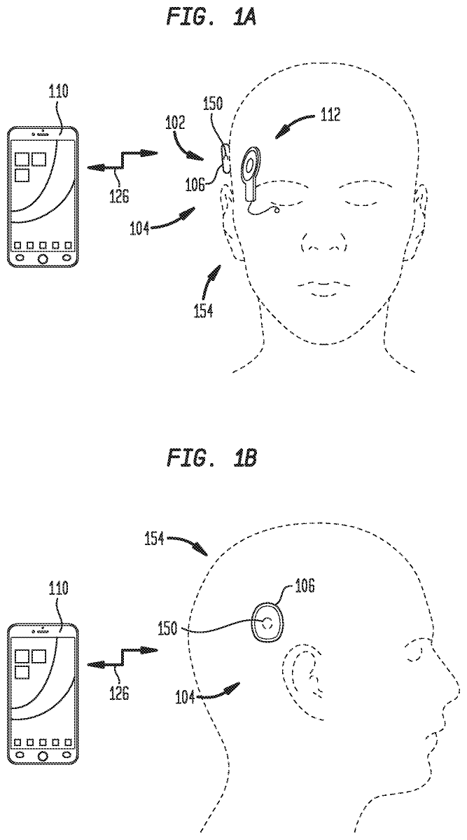

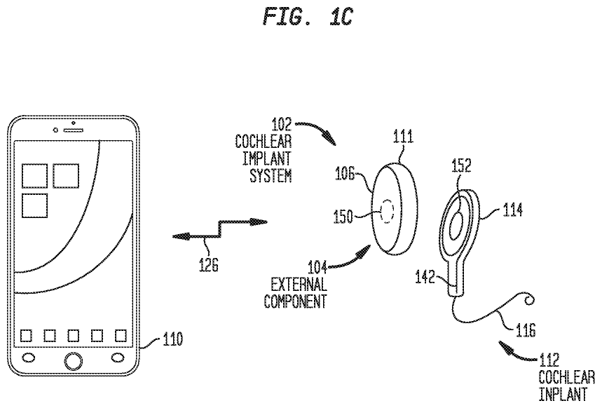

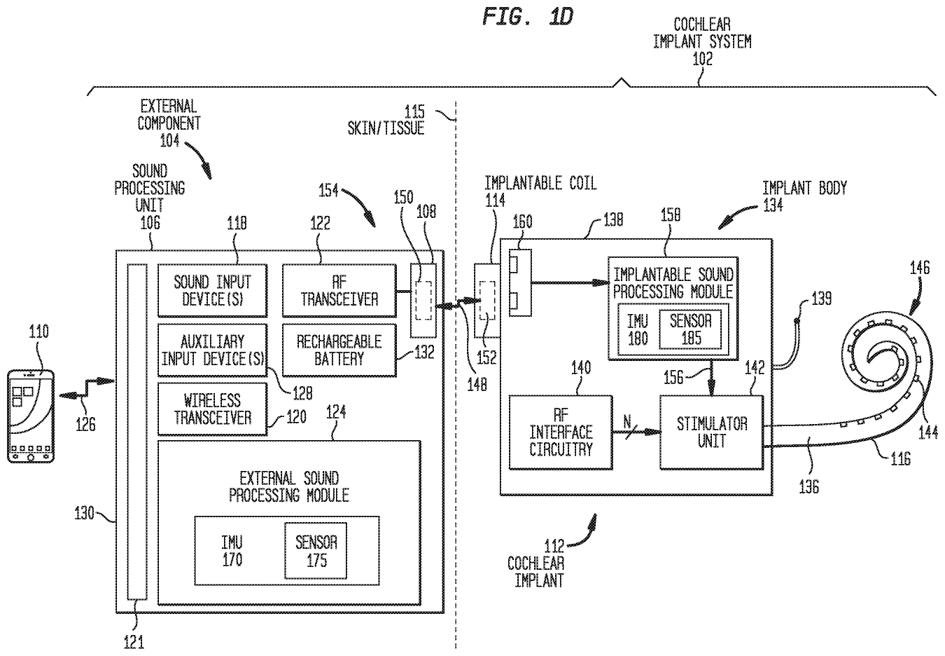

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, which is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a balance improvement application that is configured to implement the balance improvement and fall prevention techniques presented herein. The external device 110 and the cochlear implant system 102 (e.g., OTE sound processing unit 106 or the cochlear implant 112) wirelessly communicate via a bi-directional communication link 126 and interface 130. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 121, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

According to the techniques of the present disclosure, external sound processing module 124 may include an inertial measurement unit (IMU) 170. The inertial measurement unit 170 is configured to measure the inertia of sound processing unit 106. As such, inertial measurement unit 170 comprises one or more sensors 175 each configured to sense one or more of rectilinear or rotatory motion in the same or different axes. Examples of sensors 175 that may be used as part of inertial measurement unit 170 include accelerometers, gyroscopes, inclinometers, compasses, magnetometers, barometers, and the like. Such sensors may be implemented in, for example, micro electromechanical systems (MEMS) or with other technology suitable for the particular application, such as LIDAR.

The inertial measurement unit 170 may be disposed in the external sound processing module 124, which forms part of external component 104, which is in turn configured to be directly or indirectly attached to the body of a recipient. The attachment of the inertial measurement unit 170 to the recipient has sufficient firmness, rigidity, consistency, durability, etc. to ensure that the accuracy of output from the inertial measurement unit 170 is sufficient for use in the systems and methods described herein. For instance, the looseness of the attachment should not lead to a significant number of instances in which head movement that is consistent with a change in posture (as described below) is not identified as such nor a significant number of instances in which head movement that is inconsistent with a change in posture is not identified as such. In the absence of such an attachment, the inertial measurement unit 170 must accurately reflect the recipient's head movement using other techniques.

For completeness, it is noted that external sound processing module 124 may be embodied as a BTE sound processing module or an OTE sound processing module. Accordingly, the techniques of the present disclosure are applicable to both BTE and OTE hearing devices.

As also illustrated in FIG. 1D, a second inertial measurement unit 180 including sensors 185 is incorporated into implantable sound processing module 158 of implant body 134. Second inertial measurement unit 180 may serve as an additional or alternative inertial measurement unit to inertial measurement unit 170 of external sound processing module 124. Like sensors 175, sensors 185 may each be configured to sense one or more of rectilinear or rotatory motion in the same or different axes. Examples of sensors 185 that may be used as part of inertial measurement unit 180 include accelerometers, gyroscopes, inclinometers, compasses, magnetometers, barometers and the like. Such sensors may be implemented in, for example, MEMS or with other technology suitable for the particular application.

For hearing devices that include an implantable sound processing module, such as implantable sound processing module 158, that includes an IMU, such as IMU 180, the techniques presented herein may be implemented without an external processor. Accordingly, a hearing device that includes an implant body 134 and lacks an external component 104 may be configured to implement the techniques presented herein.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

As noted, the techniques disclosed herein provide a system for improving balance and balance confidence over a period of time, particularly (though not necessarily) for individuals having a hearing impairment and/or subclinical or preclinical balance difficulties. For example, research has shown that individuals that experience hearing loss, even relatively mild hearing loss, are more likely to have a history of falls. Accordingly, the disclosed techniques may be relevant to populations with hearing loss. Of course, the disclosed techniques are not limited to populations with hearing losses. Instead, the techniques may be applied to any individual with a balance or gait issue, even those with very mild or pre-clinical balance/gait issues. For example, individuals with mild balance problems may be unaware of the problem and unmotivated to address them. Thus, they require a different solution than those with a diagnosed balance pathology.

Also as noted, the techniques of this disclosure include several key interventions, which include tailored sensory cues to assist with immediate balance correction, and tailored training exercises to improve natural balance and provide ongoing balance improvement. These interventions may be implemented through sensors and AI algorithms or machine learning models that assess momentary and learned balance and gait characteristics of the user. These assessments may be used to determine fall risks. Progress and/or changes in balance and gait characteristics over time are tracked to monitor improvements or degradation of the user's characteristics, which may assist in motivation and feedback to users.

For example, when a user first receives a device configured to track user gait or balance characteristics, the user may undergo a learning period during which a baseline balance profile is determined for the user. Balance or gait characteristic limits, such as sway limits, may be determined from the baseline balance profile. If the user exhibits gait or balance characteristics that exceed these limits, balance or gait correction cues are triggered and provided to the user. Other uses of the baseline balance profile may include tracking how a user's balance improves or degrades over time. Accordingly, the initial baseline balance profile may be maintained for the user until, for example, the above-described balance or gait characteristics are no longer providing accurate balance or gait cues, either because the user's balance or gait has improved and the cues are no longer indicative of balance problems, or the user's balance or gait has degraded and the cues are not being provided in a way that prevents falls or other balance or gait issues.

The disclosed techniques aim to assess the risk of falls by detecting changes in gait and balance using appropriate motion sensors (e.g., sensors 175 and/or 185 described above with reference to FIG. 1D). The sensors may be housed within a head-worn device, such as a BTE or OTE cochlear implant sound processor or implant, as illustrated in FIG. 1D. Though, the techniques may also be implemented through other sensors, including dedicated sensors designed to implement the disclosed techniques, sensors in implanted medical devices, or sensors found in personal electronic devices, such as the inertial sensors in smartphones, smart watches, or other wearable electronic devices.

By tailoring the sensor cue interventions to an individual's needs, the disclosed techniques are more likely to be tolerated. It may be particularly important to tailor sensor cues in populations with mild or pre-clinical balance issues so that the sensor cue interventions are not overly invasive or pervasive. By tailoring balance cues-delivering them when needed-they are more relevant and more likely to increase the individual's short-term confidence. Accordingly, the benefits of the tailored cues may outweigh the burden of annoyance, fatigue or habituation that an individual with sub-clinical balance issues is predisposed to experience. The confidence and reassurance provided by these as-needed cues may encourage the individual to continue to be physically and socially active benefitting their auditory, cognitive and cardiovascular health.

The disclosed techniques may also leverage a computer application, such as a smartphone application or “app,” that communicates messages to the user. The application may also transmit data to a service provider, such as the provider of a cochlear implant. The data transmitted to the service provider may undergo data analysis, including being subject to machine learning models and/or AI algorithms, to implement the tailored intervention techniques disclosed herein. The messages communicated to the user may include the balance intervention sensory cues and exercise prescriptions aimed at strengthening specific muscle groups to improve the user's balance and/or gait. The application may also provide guidance on how to perform the prescribed exercises and visual representations of the individual's balance as they perform the exercises, such as a “spirit level.”

As used herein, “spirit level” refers to feedback to the user as they perform the exercise that might assist in balance adjustment and correction. Traditional spirit levels refer to the use of a bubble inside a glass tube containing colored alcohol or a mineral spirit solution to indicate the level or orientation of a tool. Markings on the tube indicate the center point. If the bubble is in the center, the tool or the surface on which the tool is placed is level. A similar concept may be used when the user is performing an exercise. A display provided on the smartphone application may indicate whether or not the user is appropriately balanced when performing the exercise. Displays analogous to the traditional spirit level may indicate if and in which direction a user is listing while performing the exercise.

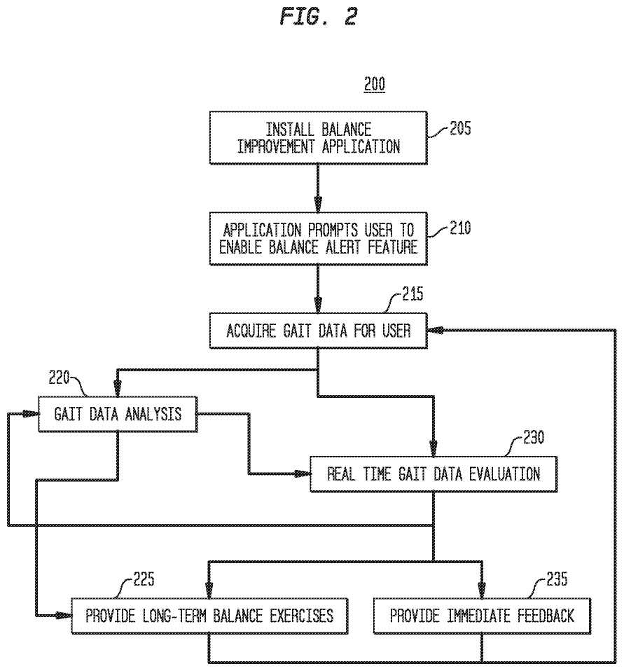

With reference now made to FIG. 2, depicted therein is a flowchart 200 illustrating a process flow for establishing a balance improvement system implementing the techniques of the present disclosure. Flowchart 200 begins in operation 205 where a balance improvement application is installed on a user device. For example, the balance improvement application may be installed on external device 110 of FIG. 1D. According to other embodiments, operation 205 may be embodied as the balance improvement application being installed on one or more of external sound processing module 124 or implantable sound processing module 158, both of FIG. 1D.

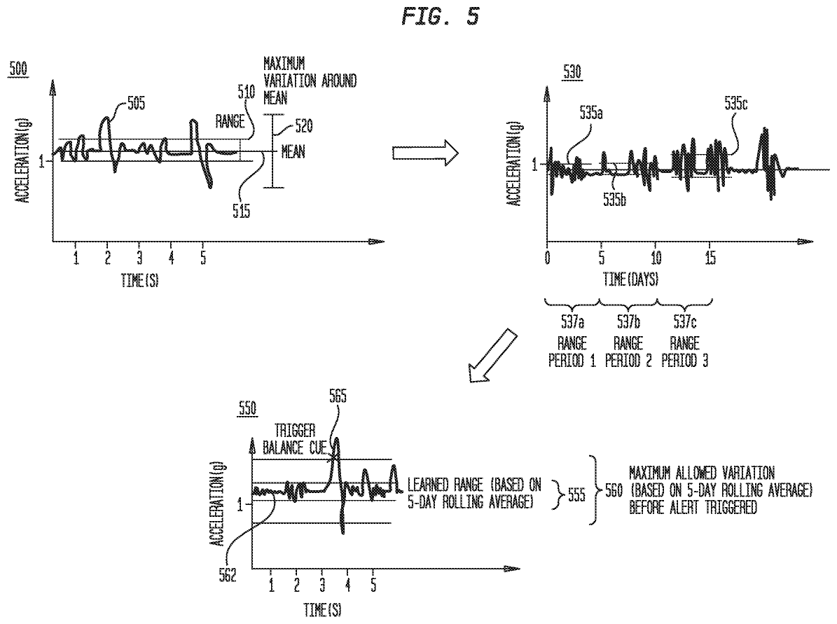

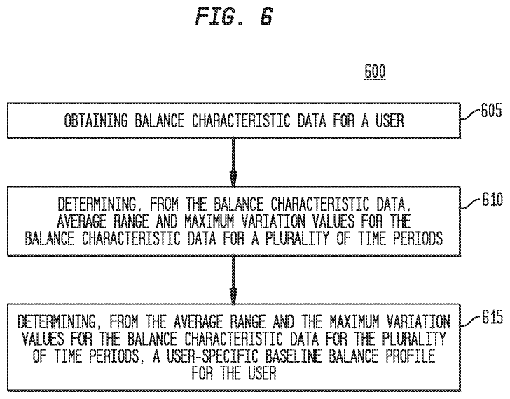

In operation 210, the balance improvement application prompts the user (e.g., a recipient of a cochlear implant) to enable a balance alert feature. By enabling the balance alert feature, gait monitoring is activated for the user, which takes place via operation 215 of flowchart 200. According to operation 215, gait or balance data may be collected by the balance improvement application. Specifically, user-worn sensors (e.g., accelerometers, gyroscopes, inclinometers, compasses, magnetometers, barometers, and the like) may collect data indicative of the user's gait or balance and transmit the data to the balance improvement application. The data collected in operation 215 may be part of an initial data collection or learning period, which is used to create a baseline balance profile for the user (as described below with reference to operation 220, as well as FIGS. 5 and 6) or part of an ongoing analysis of sensor data associated with the user to track the progression of the user's gait and balance characteristics and to provide the user with balance cues.

Once collected, the gait or balance data is analyzed in operation 220. As noted above, the analysis of the gait or balance data may include generating a baseline balance profile for the user, as will be described in detail below with reference to FIGS. 5 and 6. This baseline balance profile may be used to determine deficiencies in the user's gait and/or balance and to determine how a user's gait and balance characteristics are changing over time (as discussed below with reference to operation 225). The determination of the baseline balance profile may be performed locally at a computing device associated with the user, or the balance improvement application may send the gait or balance data to a cloud processing environment where an algorithm will assess the data to determine the baseline balance profile.

The gait or balance data provided to the cloud environment may also be made available to a health care professional, such as a physiotherapist or primary care physician. The gait or balance data may be provided to healthcare professionals via a professional portal to the cloud, via email (triggered by the user), or other means. Healthcare professionals may then use the gait or balance data to inform care or support recommendations. Additionally, medical device providers may utilize de-identified data in the cloud and be able to associate it with data logs and activity data for an individual. This use of large data sets opens the possibility to correlate balance data with other user data, for example time in speech or time speaking (e.g., own voice detection) data to assess user health in a holistic way. Further insights might be extracted from the data that might assist in future product development or in scientific publications.

According to other example embodiments, the analysis undertaken in operation 220 may be performed by the balance improvement application at an external device associated with a recipient's cochlear implant (e.g., external device 110 of FIG. 1D) or in a sound processor associated with a recipient's cochlear implant (external sound processing module 124 or implantable sound processing module 158, both of FIG. 1D).

According to specific embodiments, an algorithm applied to the gait information in operation 220 may use AI to detect and assess changes in gait and balance over short and long time periods. Based on this analysis, the process flow of flowchart 200 tailors interventions for the user. For example, in operation 225, balance exercises are provided to the user via the balance improvement application. According to embodiments in which operation 220 is performed in a service provider cloud environment, the cloud environment may communicate the long-term balance exercises to the balance improvement application executing on the user's device. The user's device will then communicate the long-term balance exercises to the user. In other embodiments, such as where the analysis of operation 220 is performed by the balance improvement application, the balance improvement application both determines and communicates the long-term balance exercises.

The long-term balance exercises of operation 225 may be tailored to identified activities of the user and areas of identified weakness for the user. For example, the analysis of operation 220 may determine from the sensor data, as well as EMAs, particular activities undertaken by the user (e.g., hiking, jogging, ice skating, aerobics, yoga, etc.) as well as particular weaknesses in the user's movement. These determinations include identifying postural transitions, changes in direction, and/or specific types of motion. Postural transitions may include rising from a prone or supine position to a sitting or standing position, and vice versa. Other postural transitions may include rising from a sitting position to a standing position, and vice versa. Determinations of changes in direction may include identifying when a user turns, while identifying specific types of motion may include identifying when a user is running, walking, walking up or down stairs, or riding on a bike. Once these types of motion are determined, specific weaknesses in the user's gait or balance may be determined. For example, weaknesses present in changes from times of inactivity to activity may be identified. The determinations may also take other factors into consideration, such as blood pressure (as measured by wearable devices, such as smart watches), low blood sugar (identified from wearable devices or specific times of day, such as immediately after waking up), and hydration levels. Additionally, the determinations may identify specific muscle groups involved in the identified activities that may need to be strengthened. For example, muscles that are typically activated in sitting or standing include leg, hip and abdominal muscles. More specifically, the identified muscles may include the quadriceps, hamstrings, rectus femoris and gluteus muscles as well as some of the core muscles and back muscles, including the erector spinae and paraspinals muscles.

The above-described determinations may be based upon AI evaluation of the gait and balance data, as well as user input to the balance improvement application. For example, when as user undertakes an activity that may result in expected gait changes, such as hiking, ice skating or exercising, the user may provide an indication that such activities are taking place via the balance improvement application. These indications may be included in data evaluated by the analysis of operation 220. Accordingly, EMA questions and responses may be incorporated into the gait and balance data analysis of operation 220 and the identification of balance exercise of operation 225.

In addition to the long-term exercises communicated to the user in operation 225, the analysis of operation 220 may be applied to real-time evaluation of gait or balance data in operation 230 to provide immediate feedback to users in operation 235. For example, the balance improvement application may evaluate gait or balance data in real time in operation 230 to determine if a user is losing balance. Specifically, the analysis of operation 220 may be provided to the balance improvement application so that the real time evaluation of the user's gait or balance data can distinguish between acceptable changes in the gait or balance data and changes that are indicative of a loss of balance. According to specific example embodiments, the baseline balance profile determined in the initial analysis performed in operation 220 is provided to the balance improvement application. Gait or balance data subsequently received by the balance improvement application may be compared with the baseline balance profile in operation 230. User motions that deviate sufficiently from the baseline balance profile may be determined to be an indication of an imminent fall or other loss of balance. If an imminent fall or loss of balance is detected, immediate feedback may be provided to the user in operation 235 to induce the user to make corrections to his or her gait or balance to avoid the fall or loss of balance. For example, the baseline balance profile may include balance or gait characteristic limits, such as sway limits. Operation 230 may determine, in real-time, if the user exhibits gait or balance characteristics that exceed these limits, resulting in immediate feedback being provided to the user in operation 235.

Immediate feedback may be given via a head-worn device (e.g., cochlear implant system 102 of FIG. 1D) and/or the balance improvement application executing on an external device. The immediate feedback may be embodied as sensory cues, such as auditory, visual, or haptic cues, which assist the user in correcting his or her balance, reducing imminent fall risk. For example, if a high fall risk is identified, a tone of a particular frequency may be applied to a particular ear of a recipient of a cochlear implant to indicate to the recipient an appropriate corrective action to avoid the imminent fall. According to other embodiments, a particular haptic pattern from a smartphone or smart watch may indicate to the user an appropriate corrective action to avoid the imminent fall.

Depending on the user, it may be beneficial to ensure that the immediate feedback of operation 235 is only provided to the user when a fall is truly imminent, such as for users who have not yet been clinically diagnosed with balance problems (e.g., users with sub-clinical balance problems). The above-described sensory cues may be intrusive in daily life. For example, ongoing auditory balance cues may be considered analogous to tinnitus, potentially disrupting social interaction and cognition. Additionally, if the cues are not actually indicative of an imminent fall, some users may habituate to the cues, ignoring the cues, rendering them useless. Accordingly, operation 235 may be tailored to ensure that cues are provided only when necessary. Therefore, user indications may be used to tailor operation 235. As noted above, the balance improvement application may be configured to allow the user to indicate when they are undertaking a particular activity. The user indications may then be used to tailor when immediate feedback is actually provided to the user. For example, a change in gait that would signal an imminent fall when simply walking may not be indicative of an imminent fall when the user is participating in a yoga class. A user indication of a yoga class would, therefore, prevent immediate feedback for a change in gait that might otherwise trigger immediate feedback during other activities.

As also illustrated in flowchart 200, the results of real-time gait or balance data evaluation operation 230 may be fed back into the gait or balance data analysis operation 220 to tailor the delivery and form of balance training exercises. By providing the results of real-time gait or balance data evaluation operation 230 to the gait or balance data analysis operation 220, operation 220 may take into consideration how often the user is being warned of an imminent fall. Such indications may assist gait or balance data analysis operation 220 in determining whether the user's gait and balance are improving or deteriorating. Based on such changes in the user's gait or balance, gait or balance data analysis operation 220 may indicate different exercises or interventions in operation 225.

Finally, flowchart 200 illustrates real-time gait or balance data evaluation operation 230 as following from user prompt operation 210. According to other embodiments, real-time gait or balance data evaluation operation 230 may be user-activated for situations where they feel unsteady or simply want the additional confidence and reassurance provided by the balance cues. Examples of such times may be rising from sitting, late in the evening, rising from a bed in the morning, or performing a new or unfamiliar activity. Similarly, real-time gait or balance data evaluation operation 230 may be user-deactivated or silenced in situations where the user does not want to be interrupted by balance cues, such as when conversing with other individuals.

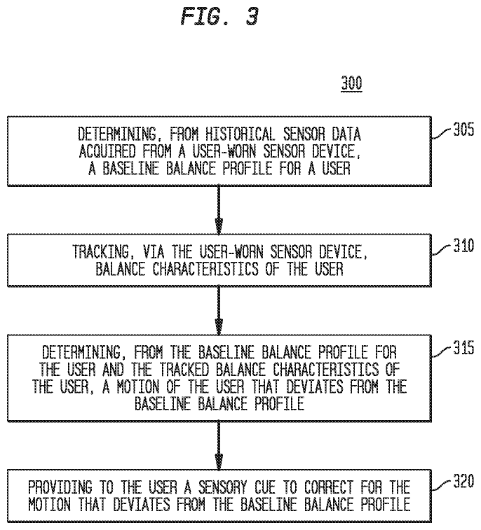

Flowchart 200 illustrates a holistic approach in which both the tailored sensory cue and tailored training exercise interventions are incorporated into a single process flow. The techniques disclosed herein are not limited to such a holistic approach. Instead, the tailored sensory cue interventions may be implemented independently from the tailored training exercise interventions, and vice versa. Accordingly, illustrated in FIG. 3 is a flowchart 300 illustrating a process flow for implementing the tailored sensory cue interventions independently from the tailored training exercise interventions.

Flowchart 300 begins in operation 305 in which a baseline balance profile is determined for a user. This baseline balance profile is determined from historical sensor data acquired from a user-worn sensor device. For example, operation 305 may be embodied as the initial analysis of a user's gait or balance data undertaken in operation 220 of FIG. 2. The sensor recited in operation 305 may be embodied as one or more of an accelerometer, a gyroscope, an inclinometer, a compass, a magnetometer, a barometer, and the like.

In operation 310, balance characteristics of the user are tracked via the user-worn sensor device. Accordingly, operation 310 may be embodied as the continued acquisition and monitoring of a user's gait or balance data that is performed in operation 215 of flowchart 200 if FIG. 2.

In operation 315, a motion of the user is determined to have deviated from the baseline balance profile for the user. This deviation is determined from the baseline balance profile for the user and the tracked balance characteristics of the user. For example, a large acceleration detected from the user-worn sensor may indicate a motion that deviates from the baseline balance profile. Such a deviation may be interpreted as being indicative of an imminent fall as it suggests a motion that is outside the user's normal balance and/or gait characteristics. Operation 315 may be embodied as the real-time gait or balance data evaluation performed by operation 230 of FIG. 2.

Finally, in operation 320, the user is provided with a sensory cue to correct for the motion that deviates from the baseline balance profile. The sensory cue may be provided by the user-worn sensor device or another device associated with the user. For example, if the user is a recipient of a cochlear implant, the sensory cue may take the form of an audio cue provided by the cochlear implant that indicates that the recipient needs to correct his or her balance or gait to prevent an otherwise imminent fall or loss of balance. According to other embodiments, the sensory cue may be provided via a haptic indication from a smartphone, smart watch, or another user-worn device. Still other embodiments may make use of visual cues, such as when a user is wearing smart glasses or is a recipient of an ocular implant. According to still other embodiments, the cues may be provided through a smart assistant (e.g., the smart assistants provided by Apple®, Amazon® and Google®). Operation 320 may be embodied as operation 235 of FIG. 2.

The use of the process flow of flowchart 300 may greatly improve user safety, confidence, and quality of life. Consider, for example, an 80-year-old female cochlear implant recipient with no diagnosed balance pathology, but who has exhibited a slow decline in confidence navigating more challenging physical tasks. While once happy to attempt new trail hikes, the recipient is now nervous and chooses to avoid such activities.

With balance cues enabled on her cochlear implant sound processor, the recipient has been gaining confidence navigating her familiar environments. On attempting a hike, she steps up a series of large, uneven, rocky steps. On one such step, with her weight transferring to her right leg on the higher rock, and there is significant sway to the right in her gait. Application of the process flow of flowchart 300 detects that this sway is outside the recipient's normal range and intervenes with an audio cue indicating the sway to the right. The recipient pauses to focus on engaging the muscles in her right leg using her arms to counterbalance and corrects the sway preventing a possible fall

The balance cue provided by the techniques presented herein allowed the recipient to both participate in the hike and instill confidence in the recipient that she will be able to navigate challenging hikes with the assistance of balance cues. Therefore, the recipient will stay active, preventing further balance and gait deterioration, and maintain her previous quality of life, increasing the recipient's satisfaction.

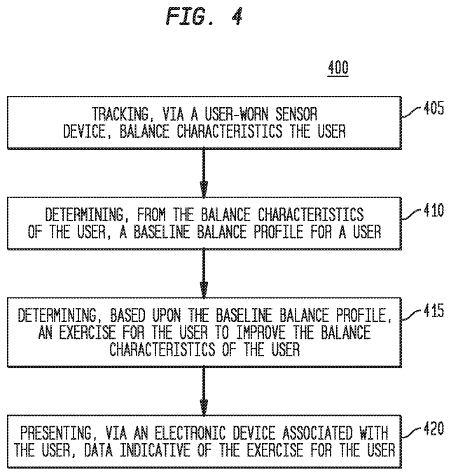

As noted above, the tailored intervention of training exercise techniques disclosed herein may also be implemented independently from the tailored sensory cue techniques. Accordingly, flowchart 400 of FIG. 4 illustrates a process flow specific to providing such tailored training exercise interventions.

Flowchart 400 begins in operation 405 in which balance characteristics of a user are tracked via a user-worn sensor device. Operation 405 may be embodied as the acquisition of user gait or balance data described above with reference to operation 215 of FIG. 2. The user-worn sensor recited in operation 405 may be embodied as one or more of an accelerometer, a gyroscope, an inclinometer, a compass, a magnetometer, a barometer, and the like.

Flowchart 400 continues in operation 410. Similar to operation 305 of FIG. 3, a baseline balance profile is determined for a user in operation 410. This baseline balance profile is determined from the balance characteristics tracked in operation 405. For example, operation 410 may be embodied as the analysis of a user's gait or balance data undertaken in operation 220 of FIG. 2.

In operation 415 an exercise for the user is determined based on the baseline balance profile. The exercise is intended to improve the user's balance characteristics. Finally, in operation 420, data indicative of the exercise for the user is presented to the user via an electronic device associated with the user. Operation 420 may be embodied as operation 225 described above with reference to FIG. 2. The electronic device recited in operation 420 may be embodied as an external device associated with a cochlear implant, such as external device 110 of FIG. 1D. The electronic device may also be embodied as a computing device, such as a smartphone device, configured with a balance improvement application. The exercises may also be presented to the user through smart glasses, and may use augmented reality to assist the user in correctly performing the exercises. For example, smart glasses may utilize the idea of a spirit-level to indicate an individual's balance as they perform the exercise.

The process flow of flowchart 400 may be applied to particular use cases to improve user outcomes and quality of life. Consider, for example, a 78-year-old male recipient of a cochlear implant with no diagnosed balance pathology who has become increasing less active. This decrease in activity may have led to a degree of muscle atrophy. The process flow of flowchart 400 has been tracking data from the recipient's sound processor (or implant) that shows a slow decrease in his stability. There have been more frequent and more dramatic swings away from his mean characteristics as a result of “wobbliness” as he goes from a sitting or lying position to a standing position. There has also been increasingly more sway in his gait when going from lengthy periods of inactivity to motion.

Through the application of the process flow of flowchart 400, the recipient's cochlear implant smartphone application alerts him that his balance has been slowly deteriorating and advises him that performing certain exercises may slow and reverse this deterioration. Following a link in his application, the recipient is presented with a personalized prescription of exercises that aim to address his observed patterns of instability. The prescription includes a sit-to-stand exercise, which is intended to increase his stability. The presentation of the exercise includes an animated image and a set of steps to inform him how to perform this exercise. There is also a link to a video with an audio description if he needs more guidance. He hits a button on the application, and as he performs the exercise the application shows him a visual representation of his balance akin to a spirit level. The application may also recommend that the recipient increases his daily active minutes, which is intended to address his “wobbliness.”

Over multiple training sessions with the balance exercises, the recipient is given feedback on his progress. This feedback shows that he is steadily improving in performing these exercises with less instability. The feedback provides an objective assessment for what the recipient is feeling-increasing strength and improvement in his balance. The objective and subjective feedback encourage him to continue with the balance training and activity. With increased activity and confidence, the recipient more frequently leaves the house to socialize. The increased social contact has a positive impact on his hearing performance and may contribute to reducing cognitive decline.

Each of the process flows of flowcharts 200, 300 and 400 of FIGS. 2-4 may include the use of a baseline balance profile. One example of the creation of such a profile is illustrated in FIG. 5. As discussed in more detail below, AI algorithms or machine learning models may be used to generate balance profile 550.

Baseline balance profile 550 includes a learned range 555 and a maximum allowed variation 560. Learned range 555 is indicative of the user's normal gait characteristics. As shown in FIG. 5, learned range 555 represents a range of acceleration values detected from a sensor associated with the user. Accordingly, the user's normal gait results in acceleration values generally within learned range 555. Maximum allowed variation 560 represents the maximum variation that is allowed before the user is presented with a sensory cue. According to the specific example of FIG. 5, an acceleration value outside of maximum allowed variation 560 may be associated with user motion indicative of an imminent fall. Therefore, when such a deviation is detected, a sensory cue is provided to the user to prompt the user to take action to prevent such a fall. As such, maximum allowed variation 560 serves as a threshold, and deviations therefrom result in sensory cues being provided to the user.

The generation of balance profile 550 is initially based on AI algorithms applied to data 500. Data 500 includes acceleration versus time (in seconds) data 505 for individual bursts of activity by a user, such as when a user goes from standing to walking. The AI algorithm determines sway range 510, mean sway 515 and maximum sway variation about the mean 520. As used herein, “sway” or “postural sway” refers to the concept of “postural stability.” Accordingly, “sway” generally refers to a measure of the amplitude of motion about a mean or center point. Therefore, a measure of sway may be a measure of the magnitude of the user's movement or swings away from an upright position. When measuring sway, sway may be described in terms of “medial-lateral sway” (side sway) and “anterior-posterior sway” (front/back sway). Sway may be measured in terms of millimeters (mm) or centimeters (cm). Determinations of sway may also include measures of acceleration of sway and peak velocity in one or more of the above-described directional planes. Measures of sway may also look at the total sway area.

Measures other than sway may also be used without deviating from the disclosed techniques. For example, if the individual is sitting and no motion is detected, then a measure of the user's lean angle may be determined. Other measures may include the time spent sitting. Similar measures may be made based upon the user's gait. For example, measures may include gait speed or velocity (e.g., meters/sec), gait cadence (e.g., steps/min), as well as the user's distance travelled in an episode of activity. More specific measures may include gait onset velocity, mean stride velocity, the variation of the stride velocity, time taken to perform a stride, stride length, steps required to reach steady gait, and others known to the skilled artisan.

Multiple individual bursts of activity data 500 are collected over time. From this data, the AI algorithm generates rolling 5-day averages for mean sway, range of sway and maximum sway variation about the mean. As shown in rolling average data 530, average range values 535a-c are determined for acceleration vs. time (in days) data over 5-day periods 537a-c, respectively.

Finally, the AI algorithm generates individualized balance profile 550 from rolling average data 530. As illustrated in FIG. 5, subsequent to the creation of learned range 555 and maximum allowed variation 560, user gait or balance data 562 may be acquired, as described above with reference to FIGS. 2-4 above. When user gait or balance data 562 exceeds the maximum allowed variation 560, a sensory cue may be provided to the user as illustrated to through balance cue trigger 565. For example, if the user's gait or balance data 562 indicates a sway value beyond the user's usual limit, a near-instantaneous balance cue may be provided to the user. Such sway value limits may be stored in a smartphone application or in a wearable or implanted device.

Additionally, if learned range 555 expands over time, it may serve as an indication that the user's balance and/or gait characteristics are deteriorating. For example, as shown in rolling average data 530, the average range values 535a-c for the user are steadily increasing with time. This will result in an expansion in learned range 555 over time, which may trigger the tailored training exercise techniques described above with reference to FIGS. 2 and 4.

With reference now made to FIG. 6, depicted therein is a flowchart 600 illustrating a generalized process flow for the baseline balance profile generation techniques disclosed herein. Flowchart 600 begins in operation 605 in which balance characteristic data is acquired for a user. Operation 605 may be embodied as the acquisition of gait or balance data from a user-worn device, as described above with reference to FIGS. 2-5.

In operation 610, average range and maximum variation values are determined for the balance characteristic data. These average range and maximum variation values are determined for a plurality of periods of time. Operation 610 may be embodied as the generation of the data illustrated in activity data 500 and rolling average data 530 described above with reference to FIG. 5.

Finally, in operation 615, a user-specific baseline balance profile is determined for the user. This user-specific baseline balance profile is determined from the average range and the maximum variation values for the plurality of time periods.

In addition to these operations, the process flow of flowchart 600 may include additional operations. For example, additional balance characteristic data may be acquired for the user subsequent to the determination of the user-specific baseline balance profile. This data may be compared with the user-specific baseline balance profile, in real time, to determine when sensory cues should be presented to the user. As a further example, the characteristics of the user-specific baseline balance profile may be used to determine and provide user-specific exercises to the user to help the user improve his or her balance and/or gait. Furthermore, changes to the user-specific baseline balance profile over time may be used to evaluate the improvement or deterioration of the user's balance or gait, which allows modified or additional exercises to be provided to the user.

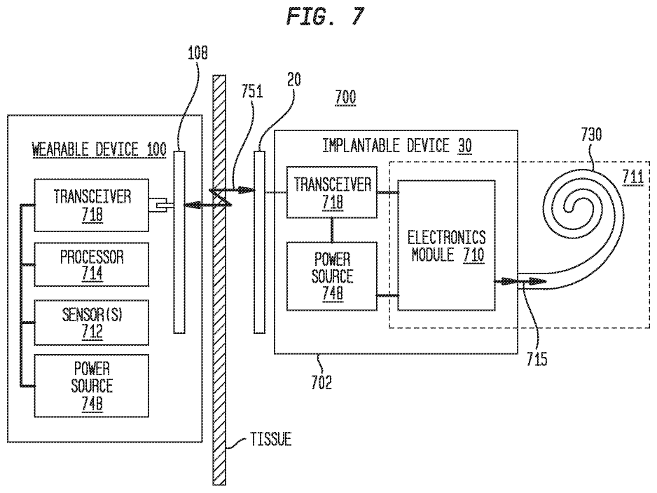

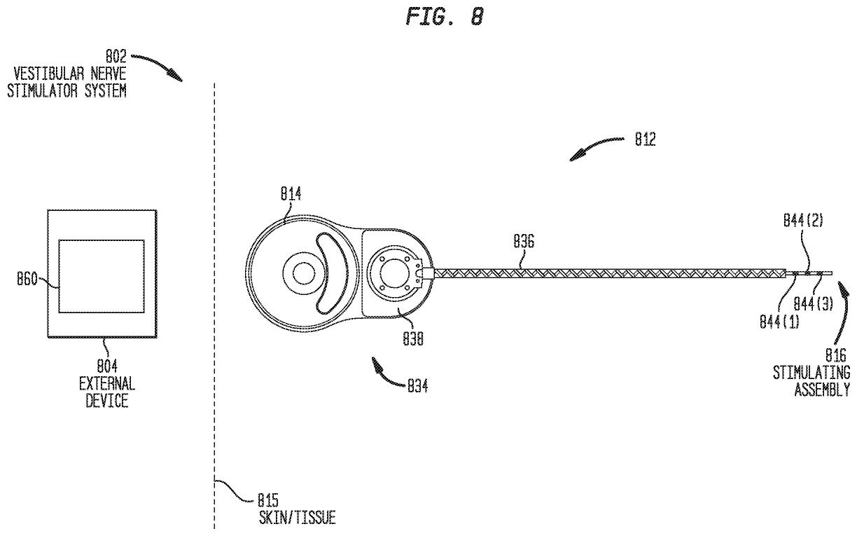

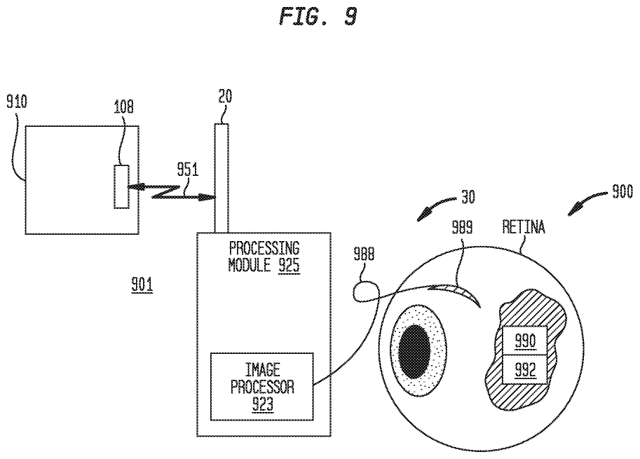

As previously described, the technology disclosed herein can be applied in any of a variety of circumstances and with a variety of different devices. Example devices that can benefit from technology disclosed herein are described in more detail in FIGS. 7-9, below. For example, the techniques described herein may be implemented through wearable medical devices, such as an implantable stimulation system as described in FIG. 7, a vestibular stimulator as described in FIG. 8, or a retinal prosthesis as described in FIG. 9. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 7 is a functional block diagram of an implantable stimulator system 700 that can benefit from the technologies described herein. The implantable stimulator system 700 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 702. Here, the wearable device 100 is configured to transcutaneously couple with the implantable device 30 via a wireless connection to provide additional functionality to the implantable device 30.

In the illustrated example, the wearable device 100 includes one or more sensors 712, a processor 714, a transceiver 718, and a power source 748. The one or more sensors 712 can be one or more units configured to produce data based on sensed activities. In an example where the stimulation system 700 is an auditory prosthesis system, the one or more sensors 712 include sound input sensors, such as a microphone, an electrical input for an FM hearing system, other components for receiving sound input, or combinations thereof. Where the stimulation system 700 is a visual prosthesis system, the one or more sensors 712 can include one or more cameras or other visual sensors. Where the stimulation system 700 is a cardiac stimulator, the one or more sensors 712 can include cardiac monitors. The processor 714 can be a component (e.g., a central processing unit) configured to control stimulation provided by the implantable device 30. The stimulation can be controlled based on data from the sensor 712, a stimulation schedule, or other data. Where the stimulation system 700 is an auditory prosthesis, the processor 714 can be configured to convert sound signals received from the sensor(s) 712 (e.g., acting as a sound input unit) into signals 751. The transceiver 718 is configured to send the signals 751 in the form of power signals, data signals, combinations thereof (e.g., by interleaving the signals), or other signals. The transceiver 718 can also be configured to receive power or data. Stimulation signals can be generated by the processor 714 and transmitted, using the transceiver 718, to the implantable device 30 for use in providing stimulation.

Sensors 712 may also include an IMU, analogous to IMU 170 or IMU 180 of FIG. 1D. Accordingly, sensors 712 may include accelerometers, gyroscopes, inclinometers, compasses, magnetometers, barometers and the like.

In the illustrated example, the implantable device 30 includes a transceiver 718, a power source 748, and a medical instrument 711 that includes an electronics module 710 and a stimulator assembly 730. The implantable device 30 further includes a hermetically sealed, biocompatible implantable housing 702 enclosing one or more of the components.

The electronics module 710 can include one or more other components to provide medical device functionality. In many examples, the electronics module 710 includes one or more components for receiving a signal and converting the signal into the stimulation signal 715. The electronics module 710 can further include a stimulator unit. The electronics module 710 can generate or control delivery of the stimulation signals 715 to the stimulator assembly 730. In examples, the electronics module 710 includes one or more processors (e.g., central processing units or microcontrollers) coupled to memory components (e.g., flash memory) storing instructions that when executed cause performance of an operation. In examples, the electronics module 710 generates and monitors parameters associated with generating and delivering the stimulus (e.g., output voltage, output current, or line impedance). In examples, the electronics module 710 generates a telemetry signal (e.g., a data signal) that includes telemetry data. The electronics module 710 can send the telemetry signal to the wearable device 100 or store the telemetry signal in memory for later use or retrieval.

Electronics module 710 may also include an IMU, analogous to IMU 170 or IMU 180 of FIG. 1D. Accordingly, electronics module 710 may include accelerometers, gyroscopes, inclinometers, compasses, magnetometers, barometers and the like.

The stimulator assembly 730 can be a component configured to provide stimulation to target tissue. In the illustrated example, the stimulator assembly 730 is an electrode assembly that includes an array of electrode contacts disposed on a lead. The lead can be disposed proximate tissue to be stimulated. Where the system 700 is a cochlear implant system, the stimulator assembly 730 can be inserted into the recipient's cochlea. The stimulator assembly 730 can be configured to deliver stimulation signals 715 (e.g., electrical stimulation signals) generated by the electronics module 710 to the cochlea to cause the recipient to experience a hearing percept. In other examples, the stimulator assembly 730 is a vibratory actuator disposed inside or outside of a housing of the implantable device 30 and configured to generate vibrations. The vibratory actuator receives the stimulation signals 715 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 718 can be components configured to transcutaneously receive and/or transmit a signal 751 (e.g., a power signal and/or a data signal). The transceiver 718 can be a collection of one or more components that form part of a transcutaneous energy or data transfer system to transfer the signal 751 between the wearable device 100 and the implantable device 30. Various types of signal transfer, such as electromagnetic, capacitive, and inductive transfer, can be used to usably receive or transmit the signal 751. The transceiver 718 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 748 can be one or more components configured to provide operational power to other components. The power source 748 can be or include one or more rechargeable batteries. Power for the batteries can be received from a source and stored in the battery. The power can then be distributed to the other components as needed for operation.

As should be appreciated, while particular components are described in conjunction with FIG. 7, technology disclosed herein can be applied in any of a variety of circumstances. The above discussion is not meant to suggest that the disclosed techniques are only suitable for implementation within systems akin to that illustrated in and described with respect to FIG. 7. In general, additional configurations can be used to practice the methods and systems herein and/or some aspects described can be excluded without departing from the methods and systems disclosed herein.

FIG. 8 illustrates an example vestibular stimulator system 802, with which embodiments presented herein can be implemented. As shown, the vestibular stimulator system 802 comprises an implantable component (vestibular stimulator) 812 and an external device/component 804 (e.g., external processing device, battery charger, remote control, etc.). The external device 804 comprises a transceiver unit 860. As such, the external device 804 is configured to transfer data (and potentially power) to the vestibular stimulator 812. External device 804 may also include an IMU, analogous to IMU 170 or IMU 180 of FIG. 1D. Accordingly, external device 804 may include accelerometers, gyroscopes, inclinometers, compasses, magnetometers, barometers and the like.

The vestibular stimulator 812 comprises an implant body (main module) 834, a lead region 836, and a stimulating assembly 816, all configured to be implanted under the skin/tissue (tissue) 815 of the recipient. The implant body 834 generally comprises a hermetically-sealed housing 838 in which RF interface circuitry, one or more rechargeable batteries, one or more processors, and a stimulator unit are disposed. The implant body 134 also includes an internal/implantable coil 814 that is generally external to the housing 838, but which is connected to the transceiver via a hermetic feedthrough (not shown).

Implant body 814 may also include an IMU, analogous to IMU 170 or IMU 180 of FIG. 1D. Accordingly, implant body 814 may include accelerometers, gyroscopes, inclinometers, compasses, magnetometers, barometers and the like.

The stimulating assembly 816 comprises a plurality of electrodes 844(1)-(3) disposed in a carrier member (e.g., a flexible silicone body). In this specific example, the stimulating assembly 816 comprises three (3) stimulation electrodes, referred to as stimulation electrodes 844(1), 844(2), and 844(3). The stimulation electrodes 844(1), 844(2), and 844(3) function as an electrical interface for delivery of electrical stimulation signals to the recipient's vestibular system.

The stimulating assembly 816 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

In operation, the vestibular stimulator 812, the external device 804, and/or another external device, can be configured to implement the techniques presented herein. That is, the vestibular stimulator 812, possibly in combination with the external device 804 and/or another external device, can include an evoked biological response analysis system, as described elsewhere herein.

FIG. 9 illustrates a retinal prosthesis system 901 that comprises an external device 910 (which can correspond to the wearable device 100) configured to communicate with a retinal prosthesis 900 via signals 951. The retinal prosthesis 900 comprises an implanted processing module 925 (e.g., which can correspond to the implantable device 30) and a retinal prosthesis sensor-stimulator 990 is positioned proximate the retina of a recipient. The external device 910 and the processing module 925 can communicate via coils 108, 20.

External device 910 may also include an IMU, analogous to IMU 170 or IMU 180 of FIG. 1D. Accordingly, external device 910 may include accelerometers, gyroscopes, inclinometers, compasses, magnetometers, barometers and the like.

In an example, sensory inputs (e.g., photons entering the eye) are absorbed by a microelectronic array of the sensor-stimulator 990 that is hybridized to a glass piece 992 including, for example, an embedded array of microwires. The glass can have a curved surface that conforms to the inner radius of the retina. The sensor-stimulator 990 can include a microelectronic imaging device that can be made of thin silicon containing integrated circuitry that convert the incident photons to an electronic charge.

The processing module 925 includes an image processor 923 that is in signal communication with the sensor-stimulator 990 via, for example, a lead 988 which extends through surgical incision 989 formed in the eye wall. In other examples, processing module 925 is in wireless communication with the sensor-stimulator 990. The image processor 923 processes the input into the sensor-stimulator 990 and provides control signals back to the sensor-stimulator 990 so the device can provide an output to the optic nerve. That said, in an alternate example, the processing is executed by a component proximate to, or integrated with, the sensor-stimulator 990. The electric charge resulting from the conversion of the incident photons is converted to a proportional amount of electronic current which is input to a nearby retinal cell layer. The cells fire and a signal is sent to the optic nerve, thus inducing a sight perception.

The processing module 925 can be implanted in the recipient and function by communicating with the external device 910, such as a behind-the-ear unit, a pair of eyeglasses, etc. The external device 910 can include an external light/image capture device (e.g., located in/on a behind-the-ear device or a pair of glasses, etc.), while, as noted above, in some examples, the sensor-stimulator 990 captures light/images, which sensor-stimulator is implanted in the recipient.

Processing module 925 may also include an IMU, analogous to IMU 170 or IMU 180 of FIG. 1D. Accordingly, processing module 925 may include accelerometers, gyroscopes, inclinometers, compasses, magnetometers, barometers and the like.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method comprising:
determining, from historical sensor data acquired from a user-worn sensor device, a baseline balance profile for a user;
tracking, via the user-worn sensor device, balance characteristics of the user;
determining, from the baseline balance profile for the user and the tracked balance characteristics of the user, a motion of the user that deviates from the baseline balance profile; and
providing to the user a sensory cue to correct for the motion that deviates from the baseline balance profile.

2. The method of claim 1, wherein determining the baseline balance profile for the user comprises determining the baseline balance profile using an artificial intelligence algorithm.

3. The method of claim 1, wherein the balance characteristics comprise a gait characteristic.

4. The method of claim 1, wherein the user-worn sensor device comprises a head-worn device.

5. (canceled)

6. (canceled)

7. The method of claim 1, wherein determining the motion of the user that deviates from the baseline balance profile comprises:
tracking feedback from the user in response to providing the user with the sensory cue; and
adjusting a threshold deviation from the baseline balance profile that will trigger the providing of the sensory cue to the user.

8. The method of claim 7, further comprising:
receiving the feedback from the user via the user-worn sensor device in response to providing the user the sensory cue.

9. The method of claim 7, wherein the feedback comprises the user silencing the sensory cue in response to providing the user the sensory cue.

10. The method of claim 1, further comprising modifying the baseline balance profile for the user in response to the tracking the balance characteristics of the user.

11. The method of claim 10, further comprising:
determining, from the modifying the baseline balance profile for the user, a degradation of a physical characteristic of the user;
determining an exercise for the user to improve the physical characteristic of the user; and
presenting, via an electronic device associated with the user, data indicative of the exercise for the user.

12. The method of claim 11, further comprising:
tracking balance characteristics of the user as the user performs the exercise;
determining, from the balance characteristics of the user as the user performs the exercise, feedback indicating how well the user is performing the exercise; and
presenting to the user, via the electronic device associated with the user, data indicative of the feedback indicating how well the user is performing the exercise.

13. The method of claim 11, further comprising:
modifying, subsequent to presenting the data indicative of the exercise, the baseline balance profile for the user; and
providing to the user, via the electronic device, data indicative of feedback indicating an effect of the exercise on the baseline balance profile.

14-38. (canceled)

39. A system comprising:
a user worn sensor device configured to acquire data associated with balance or gait characteristics of a user;
a user interface device; and
a processing device comprising one or more processors configured to:
receive, from the user worn sensor device, first data associated with the balance or gait characteristics of the user;
determine, from the first data, a baseline balance profile for a user;
receive, via the user worn sensor device, second data associated with the balance or gait characteristics of the user;
determine, from the baseline balance profile for the user and the second data, a motion of the user that deviates from the baseline balance profile; and
provide to the user, via the user interface device, a sensory cue to correct for the motion that deviates from the baseline balance profile.

40. The system of claim 39, wherein the one or more processors are configured to determine the baseline balance profile for the user by determining the baseline balance profile using an artificial intelligence algorithm.

41. The system of claim 39, wherein the user worn sensor device comprises a head-worn device.

42. The system of claim 39, wherein the user worn sensor device comprises a hearing device.

43. The system of claim 39, wherein the user worn sensor device comprises an implantable medical device.

44. The system of claim 39, wherein the one or more processors are configured to determine the motion of the user that deviates from the baseline balance profile by:
tracking, via the user interface device, feedback from the user in response to providing the user with the sensory cue; and
adjust a threshold deviation from the baseline balance profile that will trigger the providing of the sensory cue to the user.

45. The system of claim 44, wherein the feedback comprises the user silencing the sensory cue in response to providing the user the sensory cue.

46. The system of claim 39, wherein the one or more processors are configured to modify the baseline balance profile for the user in response to receiving the second data.

47. The system of claim 46, wherein the one or more processors are further configured to:
determine, from the modifying the baseline balance profile for the user, a degradation of a physical characteristic of the user;
determine an exercise for the user to improve the physical characteristic of the user; and
present, via the user interface device, data indicative of the exercise for the user.

48. The system of claim 47, wherein the one or more processors are further configured to:
receive, via user worn sensor device, third data indicative of the balance or gait characteristics of the user as the user performs the exercise;
determine, from the third data, feedback indicating how well the user is performing the exercise; and
present to the user, via the user interface device, data indicative of the feedback indicating how well the user is performing the exercise.

49. The system of claim 47, wherein the one or more processors are further configured to:
modify, subsequent to presenting the data indicative of the exercise, the baseline balance profile for the user; and
provide to the user, via the user interface device, data indicative of feedback indicating an effect of the exercise on the baseline balance profile.

50. One or more non-transitory computer readable storage media comprising instructions that, when executed by one or more processors, cause the one or more processors to:
track, via a user-worn sensor device, balance characteristics of a user;
determine, from the balance characteristics of the user, a baseline balance profile for a user;
determine, based on the baseline balance profile, an exercise for the user to improve the balance characteristics of the user; and
present, via an electronic device associated with the user, data indicative of the exercise for the user.

51. The one or more non-transitory computer readable storage media of claim 50, wherein the baseline balance profile is determined using an artificial intelligence algorithm.

52. The one or more non-transitory computer readable storage media of claim 50, further comprising instructions that, when executed by one or more processors, cause the one or more processors to:
track balance characteristics of the user as the user performs the exercise;
determine, from the balance characteristics of the user as the user performs the exercise, feedback indicating how well the user is performing the exercise; and
present to the user, via the electronic device associated with the user, data indicative of the feedback indicating how well the user is performing the exercise.

53. The one or more non-transitory computer readable storage media of claim 50, further comprising instructions that, when executed by one or more processors, cause the one or more processors to:
modify, subsequent to presenting the data indicative of the exercise, the baseline balance profile for the user; and
provide to the user, via the electronic device, data indicative of feedback indicating an effect of the exercise on the baseline balance profile.

54. The one or more non-transitory computer readable storage media of claim 50, wherein the user-worn sensor device comprises a head-worn device.

55. The one or more non-transitory computer readable storage media of claim 50, wherein the user-worn sensor device comprises at least one of a hearing device or an implantable medical device.
